(19) United States
(12) Patent Application Publication
(10) Pub. No.: US 2023/0193399 A1
(43) Pub. Date: Jun. 22, 2023

(54) TUMOR MUTATIONAL BURDEN ASSOCIATED WITH SENSITIVITY TO IMMUNOTHERAPY IN LOCALLY ADVANCED OR METASTATIC UROTHELIAL CARCINOMA

(71) Applicant: ASTRAZENECA AB, Sodertale (SE)

(72) Inventors: Han SI, Wilmington, DE (US); Michael KUZIORA, Wilmington, DE (US); Brandon W. HIGGS, Wilmington, DE (US)

(21) Appl. No.: 17/926,235

(22) PCT Filed: May 21, 2021
(86) PCT No.: PCT/EP2021/063659

§ 371 Date: Nov. 18, 2022

Related U.S. Application Data
(60) Provisional application No. 63/028,070, filed on May 21, 2020.

(51) Int. Cl. C12Q 1/6886; C07K 16/28; A61P 35/00; A61P 13/02

(52) U.S. Cl. CPC C12Q 1/6886; A61P 13/02; A61P 35/00; C07K 16/2827; C12Q 2600/106; C12Q 2600/156; C12Q 2600/158

(57) ABSTRACT

The disclosure generally relates to methods for treating urothelial carcinoma patients based on use of PD-L1 expression, blood-based tumor mutation burden, and identification of mutations in circulating tumor DNA associated with sensitivity or resistance to immunotherapy to predict overall survival in patients treated with durvalumab.

### FIELD OF THE DISCLOSURE

The disclosure generally relates to methods for treating urothelial carcinoma patients based on use of PD-L1 expression, blood-based tumor mutation burden, and identification of mutations in circulating tumor DNA associated with sensitivity or resistance to immunotherapy to predict overall survival in patients treated with durvalumab.

### BACKGROUND OF THE DISCLOSURE

Urothelial carcinoma (UC) is a promising target for immune checkpoint inhibitors (ICIs) because it is particularly reliant on the programmed cell death-1 (PD-1)/programmed cell death ligand-1 (PD-L1) pathway to evade the immune system. UC also demonstrates relatively high PD-L1 expression compared to other tumors. In patients with advanced UC, clinical activity and acceptable safety have been observed with several anti-PD-1/PD-L1 agents, including the PD-L1 antagonist durvalumab.

Tumor mutational burden (TMB) has been identified as a predictive biomarker for response to immunotherapy in a number of cancers, but it is still unclear whether somatic mutations in specific genes can drive sensitivity to immunotherapy. Somatic mutations detected in circulating tumor DNA (ctDNA) can be used to assess disease progression, response to therapy, and clonality of primary and metastatic lesions. The copy number burden of interferon-gamma pathway genes, a measure of immune infiltration, has also been identified as a biomarker of response to immunotherapies.

Aberrant signaling via Fibroblast Growth Factor Receptor 3 (FGFR3) promotes cellular proliferation, survival, migration, and differentiation, leading to tumorigenesis. FGFR3 alterations in UCs tend to be driver mutations. A study of erdafitinib, a tyrosine kinase inhibitor with activity against FGFR1-4, showed an objective response in 40% of previously treated patients with locally advanced/metastatic UC who had FGFR alterations and has been approved by the U.S. Food and Drug Administration (FDA) for this indication. Earlier clinical studies of FGFR3 inhibitors also showed encouraging results in some patients, but the mechanisms of response are not well understood and there is a need for additional biomarkers to refine patient selection.

### SUMMARY OF THE DISCLOSURE

The disclosure provides a method of predicting success of a cancer treatment in a patient in need thereof, comprising determining the expression of programmed death-ligand 1 (PD-L1) on the patient’s tumor cells (TCs) and immune cells (ICs), wherein high PD-L1 expression predicts success of the treatment.

The disclosure further provides a method of predicting success of a cancer treatment in a patient in need thereof, comprising determining if the patient has a somatic mutation in fibroblast growth factor receptor 3 (FGFR3) gene, wherein a lack of a somatic mutation in FGFR3 gene predicts success of the treatment.

The disclosure further provides a method of predicting success of a cancer treatment in a patient in need thereof, comprising determining if the patient has a somatic mutation in at least one of AT-rich interactive domain-containing protein 1A gene (ARID1A) or notch receptor 1(NOTCH1) gene, wherein a somatic mutation in at least one of ARID1A or NOTCH1 gene predicts success of the treatment.

The disclosure further provides a method of treating cancer in a patient in need thereof, comprising: (a) determining if the patient has a somatic mutation in fibroblast growth factor receptor 3 (FGFR3) gene; and (b) treating or continuing treatment if patient lacks a somatic mutation in FGFR3 gene or discontinuing treatment if the patient has a somatic mutation in FGFR3 gene.

The disclosure further provides a method of treating cancer in a patient in need thereof, comprising: (a) determining whether the patient has a somatic mutation in at least one of A-T-rich interactive domain-containing protein 1A gene (ARID1A) or notch receptor 1(NOTCH1) gene; and (b) treating or continuing treatment if patient has a somatic mutation in at least one of ARID1A or NOTCH 1 gene.

Specific embodiments of the claimed invention will become evident from the following more detailed description of certain embodiments and the claims.

### BRIEF DESCRIPTION OF THE DRAWINGS

FIG. 1A shows activity by PD-L1 expression for time to response and duration of response by blinded independent central review (BICR) for ≥2 L prior platinum population.

FIG. 1B shows antitumor activity by PD-L1 expression for best percentage change from baseline in tumor size by BICR, and percentage of patients with any tumor shrinkage from baseline^{†}in the ≥2 L prior platinum population (patients with target lesions at baseline and ≥1 post-baseline scan)^{‡} *PD-L1 expression status was unknown (due to insufficient tumor in biopsy) or unavailable (as testing had not been processed at data cutoff) for 13 patients; ^{†}Percentage of patients with any tumor size reduction (excluding patients with no post-baseline tumor assessment); ^{‡}For patients with lymph nodes included in their target lesions, CR may not equate with a -100% change from baseline according to RECIST v1.1.

FIG. 2 shows Kaplan-Meier estimates of overall survival (OS) by PD-L1 expression for ≥2 L prior platinum population.

FIG. 3A shows Kaplan-Meier estimate of OS probability in patients stratified by FGFR3 mutation status.

FIG. 3B shows Kaplan-Meier estimate of OS probability in patients stratified by FGFR3 mutation status and PD-L1 expression.

FIG. 4 shows Kaplan-Meier estimate of OS probability in patients stratified by tumor mutational burden (TMB) status.

FIG. 5A and FIG. 5B show Kaplan-Meier estimates of OS probability in UC patients stratified by ARID1A or NOTCH1 mutation status respectively.

### DETAILED DESCRIPTION OF THE DISCLOSURE

The present disclosure generally relates to methods for treating urothelial carcinoma patients based on use of PD-L1 expression, blood-based tumor mutation burden, and identification of mutations in circulating tumor DNA associated with sensitivity or resistance to immunotherapy to predict overall survival in patients treated with durvalumab.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In some embodiments provided herein is a method of predicting success of a cancer treatment in a patient in need thereof, comprising determining the expression of programmed death-ligand 1 (PD-L1) on the patient’s tumor cells (TCs) and immune cells (ICs), wherein high PD-L1 expression predicts success of the treatment.

In some embodiments, the high PD-L1 expression comprises expression of PD-L1 on 25% or more of TCs and/or ICs.

In some embodiments, the method of predicting success of cancer treatment further comprises determining the patient’s tumor mutational burden (TMB), wherein a high TMB further predicts success of a treatment.

“Tumor mutational burden” or “TMB” refers to the quantity of mutations found in a tumor. TMB varies among different tumor types. Some tumor types have a higher rate of mutation than others. TMB can be measured by a variety of tools known in the field. In certain embodiments, these tools are the Foundation Medicine and Guardant Health measurement tools. In other embodiments TMB can be measured with a ctDNA assay. Determining whether a tumor has high or low levels of TMB can be determined by comparison to a reference population having similar tumors and determining median or mean level of mutations/megabase (mut/Mb). In some embodiments, a high TMB is defined as ≥ 12 to ≥ 20 mutations/megabase (mut/Mb). In other embodiments, a high TMB is defined as ≥ 16 mutations/megabase (mut/Mb). In other embodiments, a high TMB is defined as ≥ 20 mutations/megabase (mut/Mb). In some embodiments, the mutations may be selected from those outlined in Table 1. In some embodiments, the mutations may be selected from those outlined in Table 1, with the proviso that the patient lacks a mutation (e.g. somatic mutation) in FGFR3 (e.g. the patient lacks the mutations “R397C”/“C>T” and “S249C”/“C>G” that are outlined in Table 1, or lacks the mutations “R397C”/“C>T”, “Fusion to TACC3 gene” and “S249C”/“C>G” that are outlined in Table 1).

Determining whether a TMB is high may vary from tumor type to tumor type. Determining whether a tumor has high or low levels of TMB can be determined by comparison to a reference population having similar tumors and determining median or mean level of mutations/megabase (mut/Mb). In some embodiments, the levels of TMB are divided as low (1-5 mutations/mb), intermediate (6-19 mutations/mb), and high (≥20 mutations/mb).

In some embodiments, the method of predicting success of a cancer treatment further comprises determining if the patient has a somatic mutation in fibroblast growth factor receptor 3 (FGFR3), wherein a lack of a somatic mutation in FGFR3 further predicts success of the treatment. In particular embodiments, determining if the patient has a somatic mutation in fibroblast growth factor receptor 3 (FGFR3) is determined using the patient’s circulating tumor DNA (ctDNA).

In some embodiments, provided herein is a method of predicting success of a cancer treatment in a patient in need thereof, comprising determining if the patient has a somatic mutation in fibroblast growth factor receptor 3 (FGFR3) gene, wherein a lack of a somatic mutation in FGFR3 gene predicts success of the treatment. In some embodiments, determining if the patient has a somatic mutation in FGFR3 gene is determined using the patient’s circulating tumor DNA (ctDNA).

The term “FGFR3” encompasses “full-length” unprocessed FGFR3 as well as any form of FGFR3 that results from processing in the cell. The term also encompasses naturally occurring variants of FGFR3, e.g., splice variants or allelic variants.

In some embodiments, provided herein is a method of predicting success of a cancer treatment in a patient in need thereof, comprising determining if the patient has a somatic mutation in at least one of AT-rich interactive domain-containing protein 1A gene (ARID1A) or notch receptor 1 (NOTCH1) gene, wherein a somatic mutation in at least one of ARID1A or NOTCH1 gene predicts success of the treatment. In some embodiments, determining if the patient has a somatic mutation in in at least one of ARID1A or NOTCH1 gene is determined using the patient’s circulating tumor DNA (ctDNA).

The term “ARID1A” encompasses “full-length” unprocessed ARID1A as well as any form of ARID1A that results from processing in the cell. The term also encompasses naturally occurring variants of ARID1A, e.g., splice variants or allelic variants.

The term “NOTCH1” encompasses “full-length” unprocessed NOTCH1 as well as any form of NOTCH1 that results from processing in the cell. The term also encompasses naturally occurring variants of NOTCH1, e.g., splice variants or allelic variants.

In some embodiments, the methods disclosed herein comprise treatment with durvalumab. The term “durvalumab,” as used herein, refers to an antibody that selectively binds PD-L1 and blocks the binding of PD-L1 to the PD-1 and CD80 receptors, as disclosed in U.S. Pat. No. 9,493,565 (wherein durvalumab is referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). Durvalumab can relieve PD-L1-mediated suppression of human T-cell activation in vitro and inhibits tumor growth in a xenograft model via a T-cell dependent mechanism.

In some embodiments, the success of a treatment is determined by an increase in overall survival as compared to standard of care. In some embodiments, the success of a treatment is determined by a favorable objective response rate (ORR). “Standard of care” (SOC) and “platinum-based chemotherapy” refer to chemotherapy treatment comprising at least one of abraxane, carboplatin, gemcitabine, cisplatin, pemetrexed, or paclitaxel. In some embodiments, the SOC comprises abraxane + carboplatin, gemcitabine + cisplatin, gemcitabine + carboplatin, pemetrexed + carboplatin, pemetrexed + cisplatin, or paclitaxel + carboplatin, and/or objective response rate.

As used herein, Overall Survival (“OS”) relates to the time-period beginning on the date of treatment until death due to any cause. OS may refer to overall survival within a period of time such as, for example, 6 months, 9 months, 12 months, 18 months, 24 months, and the like.

As used herein, ORR (“ORR”) relates to the proportion of patients with a reduction in tumor burden compared to a predefined amount. ORR may refer to ORR within a period of time such as, for example, 6 months, 9 months, 12 months, 18 months, 24 months, and the like.

In some embodiments, the patient previously received at least one line of platinum-based chemotherapy.

The term “patient” is intended to include human and non-human animals, particularly mammals. The patient may be a human.

In some embodiments, the methods disclosed herein relate to treating a patient for a tumor disorder and/or a cancer disorder. In some embodiments, the cancer is melanoma, breast cancer, pancreatic cancer, lung cancer (e.g., non-small cell lung cancer (NSCLC) and small cell lung cancer (SCLC)), hepatocellular carcinoma, cholangiocarcinoma or biliary tract cancer, gastric cancer, oesophagus cancer, head and neck cancer, renal cancer, cervical cancer, colorectal cancer, or urothelial carcinoma.

The terms “treatment” or “treat,” as used herein, refer to therapeutic treatment. Those in need of treatment include subjects having cancer. In some embodiments, the methods disclosed herein can be used to treat tumors. In other embodiments, treatment of a tumor includes inhibiting tumor growth, promoting tumor reduction, or both inhibiting tumor growth and promoting tumor reduction.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “pharmaceutical composition” or “therapeutic composition,” as used herein, refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier,” as used herein, refer to one or more formulation materials suitable for accomplishing or enhancing the delivery of one or more antibodies of the disclosure.

When used for in vivo administration, the formulations of the disclosure should be sterile. The formulations of the disclosure may be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins (2005).

The formulations can be presented in unit dosage form and can be prepared by any method known in the art of pharmacy. Actual dosage levels of the active ingredients in the formulation of the present disclosure may be varied so as to obtain an amount of the active ingredient which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose may also depend on the route of administration. For example, subcutaneous administration may require a higher dosage than intravenous administration.

Response Evaluation Criteria In Solid Tumors (RECIST) refers to a set of published rules that define when cancer patients improve, stay the same, or worsen during treatments. The types of response a patient can have are a complete response (CR), a partial response (PR), progressive disease (PD), and stable disease (SD).

The methods provided herein can be used for disease control (DC) of a tumor. Disease control can be a complete response (CR), partial response (PR), or stable disease (SD).

A “complete response” (CR) refers to the disappearance of all lesions, whether measurable or not, and no new lesions. Confirmation of a complete response can be obtained using a repeat, consecutive assessment no less than four weeks from the date of first documentation. New, non-measurable lesions preclude CR.

A “partial response” (PR) refers to a decrease in tumor burden of ≥ 50% relative to baseline. Confirmation can be obtained using a consecutive repeat assessment at least 4 weeks from the date of first documentation.

“Progressive disease” (PD) refers to an increase in tumor burden of ≥ 25% relative to the minimum recorded (nadir). Confirmation can be obtained by a consecutive repeat assessment at least 4 weeks from the date of first documentation. New, non-measurable lesions do not define PD.

In some embodiments, provided herein is a method of treating cancer in a patient in need thereof, comprising (a) determining if the patient has a somatic mutation in fibroblast growth factor receptor 3 (FGFR3) gene; (b) treating or continuing treatment if patient lacks a somatic mutation in FGFR3 gene or discontinuing treatment if the patient has a somatic mutation in FGFR3 gene. In particular embodiments, the treatment comprises treatment with durvalumab.

In some embodiments, provided herein is a method of treating cancer in a patient in need thereof, comprising: (a) determining whether the patient has a somatic mutation in at least one of AT-rich interactive domain-containing protein 1A gene (ARID1A) or notch receptor 1 (NOTCH1) gene; and (b) treating or continuing treatment if patient has a somatic mutation in at least one of ARID1A or NOTCH1 gene.

In particular embodiments, the treatment (e.g. UC treatment) comprises treatment with durvalumab.

In some embodiments, reference to a somatic mutation in FGFR3 may mean at least one of the FGFR3 mutations outlined in Table 1, e.g. “R397C”/“C>T”, “Fusion to TACC3 gene” and “S249C”/“C>G”. In some embodiments, reference to a somatic mutation in FGFR3 may mean at least two of the FGFR3 mutations outlined in Table 1, e.g. “R397C”/“C>T”, “Fusion to TACC3 gene” and “S249C”/“C>G”. In some embodiments, reference to a somatic mutation in FGFR3 may mean each of the FGFR3 mutations outlined in Table 1, e.g. “R397C”/“C>T”, “Fusion to TACC3 gene” and “S249C”/“C>G”.

### EXAMPLES

The Examples that follow are illustrative of specific embodiments of the disclosure, and various uses thereof. They are set forth for explanatory purposes only and should not be construed as limiting the scope of the claimed invention in any way.

### Example 1: FGFR3 and Durvalumab Response in Locally Advanced/Metastatic UC

### 1. Methods

### Study Design and Participants

Study 1108 was a multicenter, open-label study of patients aged 18 years or older with histologically and/or cytologically confirmed solid tumors, Eastern Cooperative Oncology Group (ECOG) performance status of 0-1, and adequate organ and bone marrow function. Patients in the UC cohort had locally advanced/metastatic disease and had progressed on, were ineligible for, or had refused any number of prior therapies. Other key eligibility criteria for the UC cohort were reported previously (Powles et al., JAMA Oncol. 3(9): e172411 (2017)). Patients were excluded if they had received any prior immunotherapy or investigational anticancer agent within the past 4 weeks (6 weeks for monoclonal antibodies) or any concurrent chemotherapy, immunotherapy, biologic, or hormonal therapy for cancer.

The first 20 patients were enrolled regardless of PD-L1 expression. However, preliminary data suggested that PD-L1 may be expressed more commonly on ICs than on TCs (Powles et al., Nature 515(7528): 558-62 (2014)). Therefore, to ensure the ability to assess the contribution of PD-L1-expressing TCs to response, subsequent patients were required to have a minimum of 5% PD-L1 expression on TC.

Durvalumab was administered intravenously at a dose of 10 mg/kg once every 2 weeks for up to 12 months or until confirmed progressive disease, initiation of another anticancer therapy, unacceptable toxicity, or withdrawal of consent. Tumor assessments were carried out at weeks 6, 12, and 16, then every 8 weeks during the treatment period; after 12 months of treatment, patients then entered follow-up and were assessed for tumors every 2 months for the first year and every 3 months thereafter. Safety assessments were carried out from the start of the study until 90 days after the last dose of durvalumab or the start of a new treatment; toxicity was graded according to the National Cancer Institute Common Terminology Criteria for Adverse Events (v4.03). Patients who developed progressive disease during follow-up were offered retreatment with durvalumab.

### Study Endpoints

The primary safety endpoints included assessment of adverse events (AEs), serious AEs, laboratory evaluations, vital signs, and physical examinations. AEs of special interest (AESIs) and immune-mediated AEs (imAEs, i.e., AESIs necessitating treatment with systemic steroids, endocrine therapy, or immunosuppressants within 30 days of onset that were consistent with an immune-mediated mechanism) were also assessed.

The primary efficacy endpoint was ORR, defined as the percentage with complete response or partial response, carried out by blinded independent central review (BICR) using Response Evaluation Criteria in Solid Tumors (RECIST v1.1). Overall survival was among the secondary endpoints. Clinical results were analyzed for all patients who received durvalumab (as-treated population) and for those who had received at least one prior line of platinum-based chemotherapy (second-line or greater post platinum subgroup, denoted as ≥2L prior platinum population).

PD-L1 expression status was determined by central laboratory testing and was derived from a fresh tumor biopsy taken during screening or from an available tumor sample taken within 6 months prior to study entry; in the case of multiple samples, the most recent evaluable sample was used. PD-L1 expression on both tumor cells (TCs) and immune cells (ICs) was assessed using the VENTANA SP263 immunohistochemical assay. Samples were considered PD-L1 high if expression was 25% or more on TCs and/or ICs, and low or negative if PD-L1 expression was less than 25% on TCs and ICs. The scoring algorithm, which combines assessment of both TC and IC PD-L1 expression, was found to be optimal in identifying patients most likely to respond to durvalumab, with a 94.9% negative predictive value (Powles et al., JAMA Oncol. 3(9): e172411 (2017)). TMB was assessed using whole exome sequencing of tumor tissue, with high TMB defined as above the median value of the distribution. Plasma samples were collected prior to treatment for ctDNA analysis using the Guardant 360 targeted gene panel. Candidate genes of interest were selected based on previous research showing improved response rates with immunotherapy, across 15 different tumor types in patients harboring nonsynonymous mutations in BRCA2, NOTCH1, ARID1A, or NFE2L2 (Table 1) (Kuziora et al., ESMO 2018. Ann. Oncol. 29 (suppl_10): x1-x10, 2018. doi: 10.1093/annonc/mdy493). Mutations in FGFR3 were also included, based on previous studies showing that mutation status may predict resistance to immunotherapy (Kilgour et al., ESMO 2018 abstract 786P, doi: 10.1093/annonc/mdw373.14; Siefker-Radtke et al., J. Clin. Oncol. 36(15_suppl), 4503 (2018)).

TABLE 1

Mutations in genes of interest

| Variant type | Gene | Chromosome | Mut_aa | Mut_nt | Indel type |
|---|---|---|---|---|---|
| Indel | ARID1A | 1 | p.Gly1847fs | GGTG>GTT | Deletion |
| SNV | ARID1A | 1 | E2246* | G>T | |
| SNV | ARID1A | 1 | E1958G | A>G | |
| Indel | ARID1A | 1 | p.Leu2258fs | CG>C | Deletion |
| SNV | ARID1A | 1 | W1545* | G>A | |
| Indel | ARID1A | 1 | p.Gly86_Gly87dup | T>TGGCGGC | Insertion |
| Indel | ARID1A | 1 | p.Pro1633fs | CCCCCC>CCCCT | Deletion |
| SNV | ARID1A | 1 | G307S | G>A | |
| SNV | ARID1A | 1 | N1502Y | A>T | |
| Indel | ARID1A | 1 | p.Gln1334dup | C>CGCA | Insertion |
| SNV | ARID1A | 1 | Y1101* | T>A | |
| SNV | ARID1A | 1 | G191D | G>A | |
| SNV | ARID1A | 1 | E1444* | G>T | |
| SNV | ARID1A | 1 | L1100V | C>G | |
| Indel | ARID1A | 1 | p.Phe1459fs | C>CA | Insertion |
| SNV | ARID1A | 1 | G240D | G>A | |
| SNV | ARID1A | 1 | C1099F | G>T | |
| Indel | ARID1A | 1 | p.Pro1632fs | GCCCCC>G | Deletion |
| SNV | ARID1A | 1 | R1276* | C>T | |
| Indel | ARID1A | 1 | p.Asp1850fs | TG>T | Deletion |
| SNV | ARID1A | 1 | S1134F | C>T | |
| SNV | ARID1A | 1 | Q479* | C>T | |
| SNV | ARID1A | 1 | P452R | C>G | |
| SNV | ARID1A | 1 | Q566* | C>T | |
| SNV | ARID1A | 1 | S409L | C>T | |
| SNV | ARID1A | 1 | Q585* | C>T | |
| SNV | ARID1A | 1 | R750* | C>T | |
| SNV | ARID1A | 1 | S744* | C>G | |
| Indel | ARID1A | 1 | p.Pro805fs | GC>G | Deletion |
| SNV | ARID1A | 1 | Y195C | A>G | |
| Indel | ARID1A | 1 | p.Arg2233fs | TGCGGCGGGCTGCCC>T | Deletion |
| SNV | ARID1A | 1 | Q1401* | C>T | |
| SNV | ARID1A | 1 | Q840* | C>T | |
| SNV | ARID1A | 1 | L1731* | T>A | |
| SNV | ARID1A | 1 | S1930* | C>G | |
| SNV | ARID1A | 1 | R1989Q | G>A | |
| SNV | BRCA2 | 13 | S2414L | C>T | |
| SNV | BRCA2 | 13 | E2558K | G>A | |
| SNV | BRCA2 | 13 | L2688F | C>T | |
| SNV | BRCA2 | 13 | S1020C | C>G | |
| SNV | BRCA2 | 13 | S3041* | C>G | |
| SNV | BRCA2 | 13 | S76* | C>G | |
| SNV | BRCA2 | 13 | E674Q | G>C | |
| SNV | BRCA2 | 13 | S1900* | C>G | |
| SNV | FGFR3 | 4 | R397C | C>T | |
| Fusion | FGFR3 | 4 | | | Fusion to TACC3 gene |
| SNV | FGFR3 | 4 | S249C | C>G | |
| SNV | NFE2L2 | 2 | T80A | T>C | |
| SNV | NFE2L2 | 2 | R34Q | C>T | |
| SNV | NFE2L2 | 2 | F39V | A>C | |
| SNV | NFE2L2 | 2 | D77Y | C>A | |
| SNV | NFE2L2 | 2 | E79Q | C>G | |
| SNV | NFE2L2 | 2 | R34P | C>G | |
| SNV | NFE2L2 | 2 | I28T | A>G | |
| SNV | NOTCH1 | 9 | E1636K | C>T | |
| Indel | NOTCH1 | 9 | p.Leu2464fs | AG>A | Deletion |
| SNV | NOTCH1 | 9 | R2104C | G>A | |
| Indel | NOTCH1 | 9 | p.Glu473fs | T>TC | Insertion |
| SNV | NOTCH1 | 9 | E242K | C>T | |
| SNV | NOTCH1 | 9 | E242* | C>A | |
| SNV | NOTCH1 | 9 | H196N | G>T | |
| SNV | NOTCH1 | 9 | C449R | A>G | |
| SNV | NOTCH1 | 9 | E1623K | C>T | |

### Statistical Analysis

All patients who received their first dose of durvalumab 30 days or more prior to the data cutoff (DCO) date of Oct. 16, 2017 were included in the safety and efficacy analyses (as-treated population). The Clopper-Pearson method was used to estimate ORR and the 95% CIs. The Kaplan-Meier method with two-sided 95% CIs determined by the Brookmeyer and Crowley method was used to estimate OS, OS rate, and cumulative incidences of AEs. Associations between tumor mutations and PD-L1 status were evaluated using Fisher’s exact text. SAS statistical software (v9.3 or above) or R (v3.5) was used for all analyses.

### 2. Results

### Demographic and Baseline Characteristics

At the DCO, 201 patients had enrolled and received treatment (as-treated population), of whom 192 were in the ≥2 L prior platinum group. Efficacy results presented here are for the ≥2 L prior platinum group; all outcomes were similar in the full as-treated population. Demographic and baseline characteristics are summarized in Table 2; all patients had metastatic disease. Median age was 67 years (range, 34-88). The majority of patients were male (71.9%) and white (70.1%). At enrollment, 67.7% of patients had visceral metastases (35.4% in the liver), and 11.5% had lymph node metastases only. PD-L1 expression was high in 51.6% of patients and low/negative in 41.7% of patients; 6.8% had unknown PD-L1 status. Demographic and baseline characteristics for the subsets of patients analyzed for TMB (n=37) and ctDNA (n=163) are shown in Table 3.

TABLE 2

Demographic and baseline characteristics of the ≥2L prior platinum population and as-treated population

| Characteristic | ≥2L prior platinum group (n=192) | As-treated population (N=201) |
|---|---|---|
| Age, years | | |
| N | 192 | 201 |
| Median | 67.0 | 67.0 |
| Min, Max | (34, 88) | (34, 88) |
| Sex, n (%) | | |
| N | 192 | 201 |
| Female | 54 (28.1) | 58 (28.9) |
| Male | 138 (71.9) | 143 (71.1) |
| Race, n (%)* | | |
| N | 174 | 182 |
| Asian | 40 (23.0) | 40 (22.0) |
| Black or African-American | 6 (3.4) | 8 (4.4) |
| White | 122 (70.1) | 128 (70.3) |
| Other | 5 (2.9) | 5 (2.7) |
| Multiple categories checked | 1 (0.6) | 1 (0.5) |
| ECOG performance status, n (%) | | |
| N | 192 | 201 |
| 0 | 62 (32.3) | 65 (32.3) |
| 1 | 130 (67.7) | 136 (67.7) |
| Baseline hemoglobin concentration, n | | |
| (%) | 192 | 201 |
| N | 147 (76.6) | 155 (77.1) |
| ≥10 g/dL | 45 (23.4) | 46 (22.9) |
| < 10 g/dL | | |
| Stage 4 at study entry, n (%) | 192 (100) | 201 (100) |
| Sites of disease at baseline, n (%)^{†} | | |
| Visceral | 130 (67.7) | 135 (67.2) |
| Liver | 68 (35.4) | 72 (35.8) |
| Lymph node only | 22 (11.5) | 24 (11.9) |
| PD-L1 expression status, n (%) | | |
| High | 99 (51.5) | 102 (50.7) |
| Low/negative | 80 (41.6) | 86 (42.8) |
| Unknown | 13 (6.8) | 13 (6.5) |
| 0 | 0 | 9 (4.5) |
| 1 | 119 (62.0) | 119 (59.2) |
| 2 | 57 (29.7) | 57 (28.4) |
| 3 | 10 (5.2) | 10 (5.0) |
| ≥4 | 6 (3.1) | 6 (3.0) |
| Carboplatin | 56 (29.002) | 58 (28.9) |
| Cisplatin | 134 (69.008) | 138 (68.7) |
| Other platinum combination^{‡} | 2 (1.0) | 2 (1.0) |

TABLE 3

Demographic and baseline characteristics of patients analyzed for TMB and ctDNA

| Characteristic | Patients analyzed for TMB | | | Patients analyzed for ctDNA | | | | | | |
|---|---|---|---|---|---|---|---|---|---|---|
| | All (n=37) | TMB high (n=19) | TMB low (n=18) | All (n=163) | FGFR3 wt (n=135) | FGFR3 mut (n=28) | NOTCH1 wt (n=154) | NOTCH1 mut (n=9) | ARID1A wt (n=120) | ARID1A mut (n=43) |
| Age, years | | | | | | | | | | |
| N | 37 | 19 | 18 | 163 | 135 | 28 | 154 | 9 | 120 | 43 |
| Median | 68 | 69 | 67 | 67 | 67 | 67.5 | 67 | 66 | 67.5 | 66 |
| Min, Max | 48, 82 | 48, 76 | 49, 82 | 34, 88 | 41, 88 | 34, 84 | 34, 88 | 48, 79 | 34, 88 | 48, 84 |
| Sex, n (%) | | | | | | | | | | |
| N | 37 | 19 | 18 | 163 | 135 | 28 | 154 | 9 | 120 | 43 |
| Female | 12 (32.4) | 7 (36.8) | 5 (27.8) | 49 (30.1) | 40 (29.6) | 9(32.1) | 45 (29,2) | 4 (44.4) | 39 (32.5) | 10 (23.3) |
| Male | 25 (67.6) | 12 (63.2) | 13 (72.2) | 114 (69.9) | 95 (70.4) | 19 (67.9) | 109 (70.8) | 5 (55.6) | 81 (67.5) | 33 (76.007) |
| Race, n (%)* | | | | | | | | | | |
| N | 31 | 16 | 15 | 146 | 121 | 25 | 139 | 7 | 107 | 39 |
| Asian | 10 (32.3) | 6 (37.5) | 4 (26.7) | 33 (22.6) | 29 (24.0) | 4(16.0) | 31 (22.3) | 2 (28.6) | 23 (21.5) | 10 (25.6) |
| Black or African-American | 0 | 0 | 0 | 6(4.1) | 6 (5.0) | 0 | 6 (4.3) | 0 | 4 (3.7) | 2(5.1) |
| White | 21 (67.7) | 10 (62.5) | 11 (73.3) | 102 (69.9) | 83 (68.6) | 19 (76.0) | 97 (69.8) | 5 (71.4) | 77 (72.0) | 25 (64.1) |
| Other | 0 | 0 | 0 | 4 (2.7) | 3 (2.5) | 1 (4.0) | 4 (2.9) | 0 | 2 (1.9) | 2(5.1) |
| Multiple categories checked | 0 | 0 | 0 | 1 (0.7) | 0 | 1 (4.0) | 1 (0.7) | 0 | 1 (0.9) | 0 |
| ECOG performance status, n (%) | | | | | | | | | | |
| N | 37 | 19 | 18 | 163 | 135 | 28 | 154 | 9 | 120 | 43 |
| 0 | 13(35.1) | 8(42.1) | 5 (27.8) | 58 (35.6) | 48 (35.6) | 10 (35.7) | 54 (35.1) | 4 (44.4) | 42 (35.0) | 16 (37.2) |
| 1 | 24 (64.9) | 11 (57.9) | 13 (72.2) | 105 (64.4) | 87 (64.4) | 18 (64.3) | 100 (64.009) | 5 (55.6) | 78 (65.0) | 27 (62.8) |
| Sites of disease at baseline, n (%)^{†} | | | | | | | | | | |
| Visceral | 20 (54.1) | 7 (36.8) | 13 (72.2) | 107 (65.6) | 85 (63.0) | 22 (78.6) | 103 (66.9) | 4 (44.4) | 80 (66.7) | 27 (62.8) |
| Liver | 10 (27.0) | 4(21.1) | 6 (33.3) | 58 (35.6) | 46 (34.1) | 12 (42.9) | 55 (35.7) | 3 (33.3) | 38 (31.7) | 20 (46.5) |
| Lymph node only | 7(18.9) | 5 (26.3) | 2(11.1) | 21 (12.009) | 19(14.1) | 2(7.1) | 20(13.0) | 1(11.1) | 12(10.0) | 9 (20.9) |
| PD-L1 expression status, n (%) | | | | | | | | | | |
| High | 17 (45.9) | 11 (57.9) | 6 (33.3) | 91 (55.8) | 81 (60.0) | 10 (35.7) | 85 (55.2) | 6 (66.7) | 64 (53.3) | 27 (62.8) |
| Low/negative | 15 (40.5) | 5 (26.3) | 10 (55.6) | 62 (38.0) | 45 (33.3) | 17 (60.007) | 61 (39.6) | 1 (11.1) | 48 (40.0) | 14 (32.6) |
| Unknown | 5(13.5) | 3(15.8) | 2(11.1) | 10 (6.1) | 9 (6.7) | 1 (3.6) | 8 (5.2) | 2 (22.2) | 8 (6.7) | 2 (4.7) |
| Prior lines of systemic therapy for inoperable metastatic disease, n (%) | | | | | | | | | | |
| 0 | 0 | 0 | 0 | 8 (4.9) | 7 (5.2) | 1 (3.6) | 8 (5.2) | 0 | 6 (5.0) | 2 (4.7) |
| 1 | 24 (64.9) | 11 (57.9) | 13 (72.2) | 98 (60.1) | 84 (62.2) | 14 (50.0) | 92 (59.7) | 6 (66.7) | 75 (62.5) | 23 (53.5) |
| 2 | 10 (27.0) | 6(31.6) | 4 (22.2) | 42 (25.8) | 33 (24.4) | 9(32.1) | 40 (26.0) | 2 (22.2) | 29 (24.2) | 13 (30.2) |
| 3 | 3(8.1) | 2(10.5) | 1 (5.6) | 10 (6.1) | 7 (5.2) | 3(10.7) | 9 (5.8) | 1 (1.1) | 6 (5.0) | 4 (9.3) |
| ≥4 | 0 | 0 | 0 | 5(3.1) | 4 (3.0) | 1 (3.6) | 5 (3.2) | 0 | 4 (3.3) | 1 (2.3) |

### Patient Disposition

Median duration of exposure was 12.0 weeks (range, 1.6-54.4) and median duration of follow-up was 16.8 months (range, 0.4-37.7) in the ≥2 L prior platinum group. As of the DCO, no patients remained on the study treatment.

### Antitumor Activity

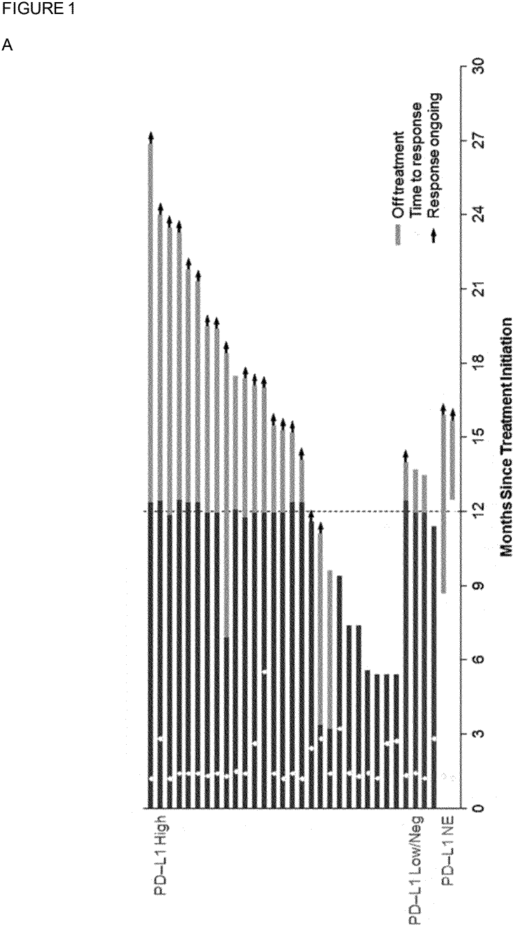

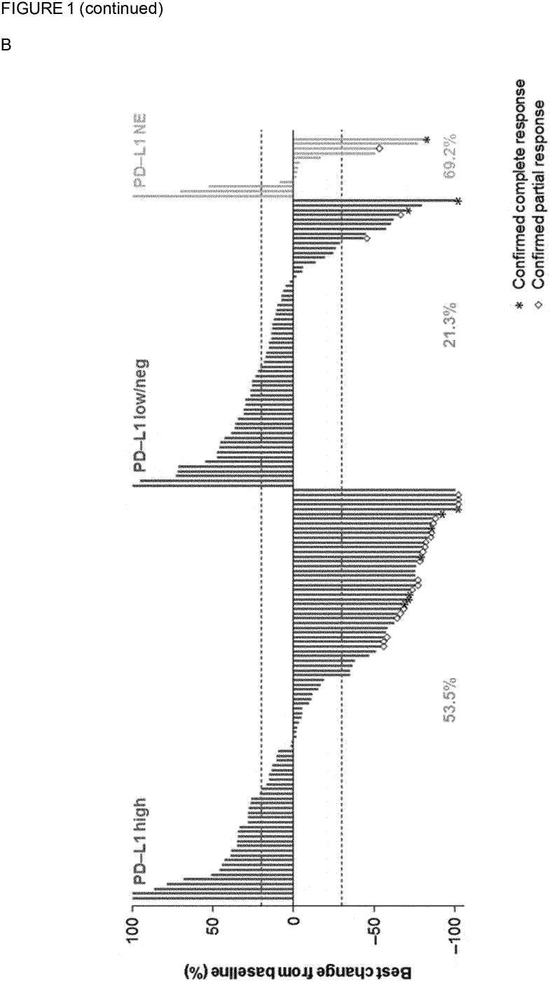

The ORR for the ≥2 L prior platinum group was 17.2% (95% CI, 12.1-23.3) for all patients; 27.3% (18.8-37.1) for the PD-L1 high subgroup and 5.0% (1.4-12.3) for the PD-L1 low/negative subgroup (Table 4). Treatment responses occurred early (FIG. 1A), with a median time to response of 1.4 months (95% CI, 1.3-1.4). Responses were durable, ranging from 2.7 to 25.7+ months, and the median duration was not reached. Responses lasted ≥6 months in 29/33 responders (87.9%) and ≥12 months in 21/33 responders (63.6%).

In the ≥2 L prior platinum group, 11/192 patients (5.7%) experienced a complete response (CR); 8/99 (8.1%) in the PD-L1 high subgroup, 2/80 (2.5%) in the PD-L1 low/negative subgroup, and 1/13 (7.7%) whose PD-L1 status was unknown. One additional CR was observed in the as-treated population in a patient with low/negative PD-L1 expression, for a total of 12/201 (6.0%) (Table 4 and FIG. 1B). Tumor shrinkage of any extent (excluding patients with no post-baseline tumor assessment) occurred in 53.5%, 21.3%, and 69.2% of patients in the PD-L1 high, PD-L1 low/negative, and PD-L1 unknown subgroups of the ≥2L prior platinum group, respectively.

The disease control rate (DCR) was 35.4% (95% CI, 28.7-42.6) in the ≥2L prior platinum group; 43.4% (33.5-53.8) in the PD-L1 high subgroup and 21.3% (12.9-31.8) in the PD-L1 low/negative subgroup (Table 4).

TABLE 4

Antitumor activity of durvalumab per BICR in the ≥2L prior platinum population and the as-treated population

| | ≥2L prior platinum group^{§} | | | As-treated population | | |
|---|---|---|---|---|---|---|
| Parameter* | Total N=192 | PD-L1 high^{‡} N=99 | PD-L1 low/negative^{‡} N=80 | Total^{†} N=201 | PD-L1 high^{‡} N=102 | PD-L1 low/negative^{‡} N=86 |
| Confirmed ORR, n (%) (95% Cl) | 33 (17.2) | 27 (27.3) | 4 (5.0) | 35 (17.4) | 28 (27.5) | 5 (5.8) |
| | (12.1, 23.3) | (18.8, 37.1) | (1.4, 12.3) | (12.4, 23.4) | (19.1, 37.2) | (1.9, 13.0) |
| CR | 11 (5.7) | 8 (8.1) | 2 (2.5) | 12 (6.0) | 8 (7.8) | 3 (3.5) |
| PR | 22 (11.5) | 19 (19.2) | 2 (2.5) | 23 (11.4) | 20 (19.6) | 2 (2.3) |
| Non-evaluable^{¶} | 32 (16.7) | 11 (11.1) | 21 (26.3) | 34 (16.9) | 17 (16.7) | 23 (16.7) |
| Responses ongoing at time of DCO | 21 (63.6) | 18 (66.7) | 1 (25.0) | 22 (62.9) | 18 (64.3) | 2 (40.0) |
| DoR, months | | | | | | |
| Median (min, max) | NR (2.7, 25.7+) | NR (2.7, 25.7+) | 12.25 (8.6, | NR (2.7, 25.7+) | NR (2.7, 25.7+) | 12.25 (8.6, 21.7+) |
| ≥6 months, n (%) | 29 (87.9) | 23 (85.2) | 12.7+) 4 (100) | 31 (88.6) | 24 (85.7) | 5 (100) |
| DCR, n (%) | 68 (35.4) | 43 (43.4) | 17 (21.3) | 72 (35.8) | 45 (44.1) | 19 (22.1) |
| (95% Cl) | (28.7, 42.6) | (33.5, 53.8) | (12.9,31.8) | (29.2, 42.9) | (34.3, 54.3) | (13.9, 32.3) |

*Based on RECIST version 1.1
^{†}Includes 14 patients who had unknown/unavailable PD-L1 status and who are not included in either the PD-L1 high or PD-L1 low/negative subgroups.
^{‡}PD-L1 expression status was unknown (due to insufficient tumor in biopsy) or unavailable (as testing had not been processed at data cutoff)
^{§}The ≥2L prior platinum subgroup includes patients who had progressed while on or after a platinum-based therapy, including those patients who progressed within 12 months of receiving therapy in a neoadjuvant/adjuvant setting processed at data cutoff) for 13 patients.
^{¶}Non-evaluable patients were those without post-baseline scans due to death, PD, or withdrawal of consent prior to the first on-treatment disease assessment or had a post-baseline scan that did not meet the minimum required interval for SD.

### Survival

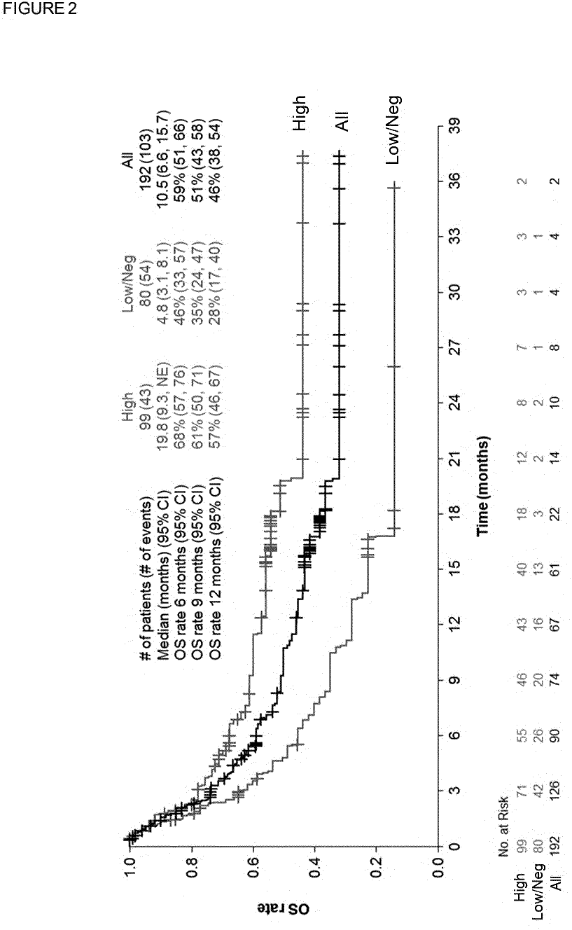

In this nonrandomized study, clinical benefit was seen across all subgroups in the ≥2L prior platinum population, regardless of baseline ECOG performance status, site of metastases, or prior lines of therapy. Median overall survival (mOS) was 10.5 months: 19.8 months for the PD-L1 high subgroup and 4.8 months for the PD-L1 low/negative subgroup (FIG. 2). The OS rates at 6, 9, and 12 months were 58.9%, 50.9%, and 46.1%, respectively.

### Safety

No new safety signals were observed. Any-grade treatment-related AEs (TRAEs) were reported in 59.7% and grade ¾ TRAEs in 9.5% of the as-treated population. The most common were fatigue (19.4%), decreased appetite (9.0%), and rash (9.0%) (Table 5). They generally occurred early. Treatment-related deaths occurred in two patients (1.0%), one due to autoimmune hepatitis and one due to pneumonitis. Six patients (3.0%) discontinued durvalumab due to TRAEs. Any-grade treatment-related AEs of special interest occurred in 36.3% of patients and were grade ¾ in 4.5%. Any-grade treatment-related imAEs were reported in 11.4% of patients and were grade ¾ in 2.0%. There were no clear differences between the PD-L1 high and PD-L1 low/negative groups in safety.

### Biomarkers

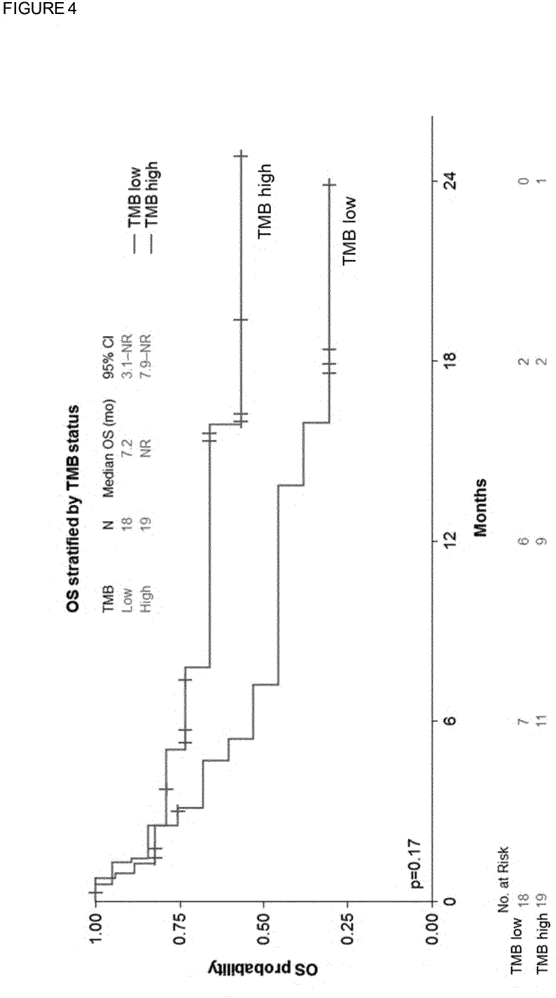

Patients with high TMB had improved OS compared to those with low TMB (hazard ratio [HR] 0.53, 95% CI, 0.2-1.5; FIG. 4).

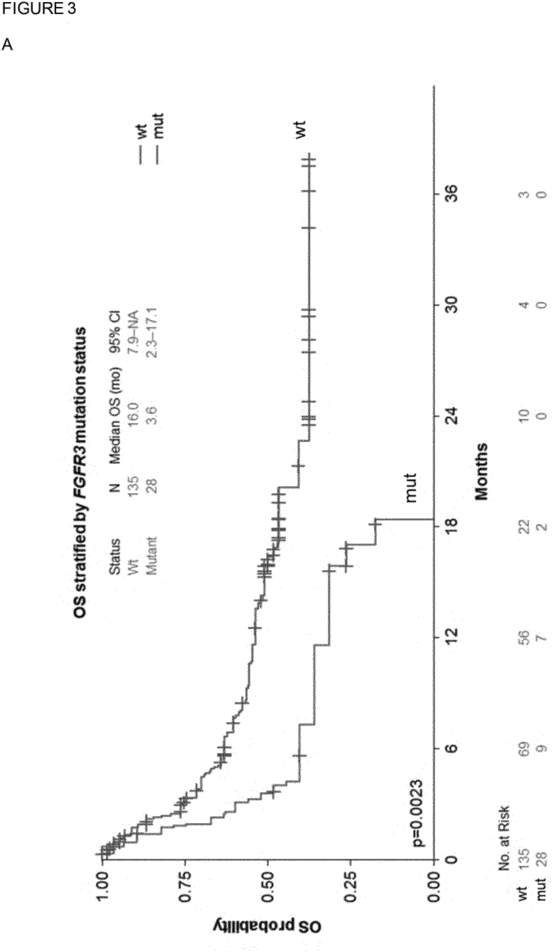

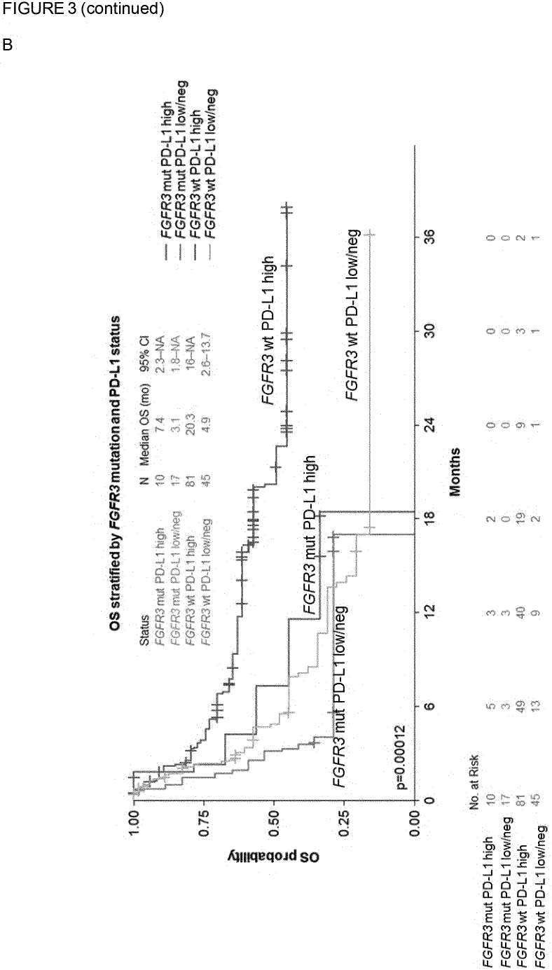

Response rates were higher in FGFR3 wt patients (20% [27/134]) than in FGFR3 mut patients (10% [3/29]). Median OS was 4.0 months in FGFR3 mut patients vs. 13.4 months in FGFR3 wt patients (HR 0.49 [95% CI, 0.3-0.79], FIG. 3A). The probability of survival was lower in FGFR3 mut patients than in FGFR3 wt patients at 12 months (0.31 [95% CI, 0.18-0.56] vs. 0.54 [95% CI, 0.45-0.63]) and at 24 months (0 vs. 0.38 [95% CI, 0.27-0.52]).

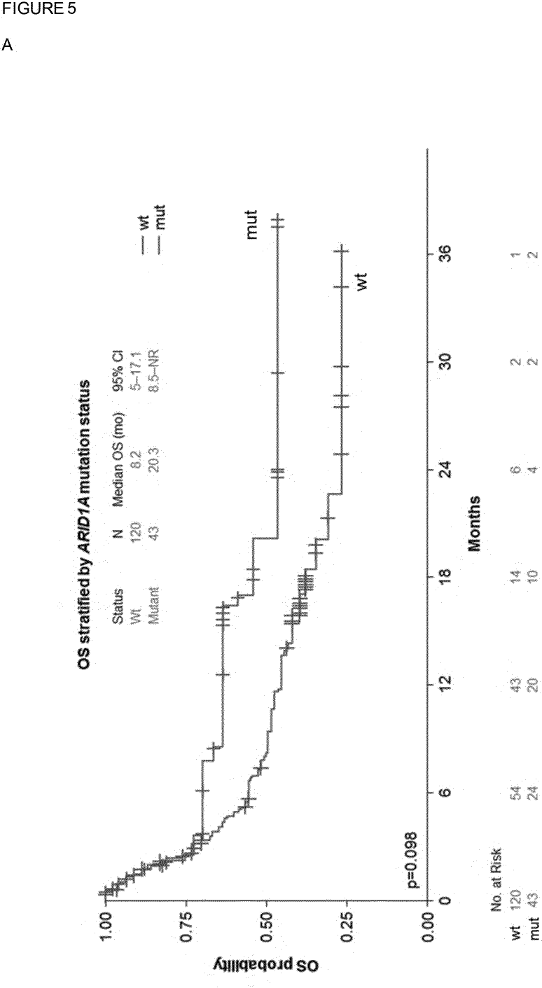

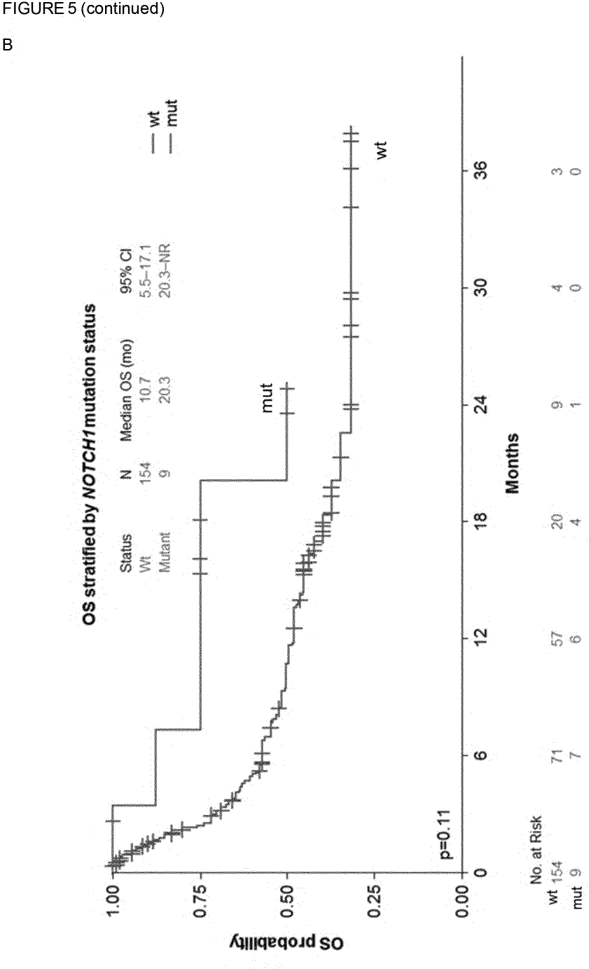

Conversely, response rates were greater in patients with nonsynonymous mutations in ARID1A than in wt patients (30% [13/43] vs. 15% [18/120]), and NOTCH1 (56% [5/9] vs. 17% [26/154]). Patients with ARIDIA and NOTCH1 mutations showed trends toward longer OS (FIG. 5).

OS was longer in patients with high PD-L1 expression than in those with low/negative expression. Notably, response rates were highest, and OS was longest (FIG. 3B) in FGFR3 wt patients with high PD-L1 expression.

In patients with known FGFR3 and PD-L1 status, PD-L1 expression was high in 81/135 (60.0%) FGFR3 wt patients and 10/28 (35.7%) FGFR3 mut patients. Fisher’s exact test and a multivariate Cox proportional hazards model showed that PD-L1 status was significantly associated with FGFR3 mutation status (p=0.01), but not with ARIDIA (p=0.36) or NOTCH1 (p=0.24) mutation status.

In patients with known FGFR3 mutation and PD-L1 status, response rates were highest in FGFR3 wt patients with PD-L1 high expression (24/80 [30.0%]) and FGFR3 mut patients with high PD-L1 expression (2/10 [20%]). Response rates were lowest in patients with low/negative PD-L1 expression (1/45 [2.2%] in FGFR3 wt patients and 1/18 [5.5%] in FGFR3 mut patients).

Among patients with high PD-L1 expression, median OS was 19.9 months for the FGFR3 wt group vs. 7.2 months for the FGFR3 mut group (HR 0.48 [95% CI 0.21-1.09]). The FGFR3 wt group included 18 patients with high TMB and 13 patients with low TMB; their median OS was not reached (95% CI 5.0-NA) and 13.7 months (95% CI 1.0-NA), respectively. There was no correlation between FGFR3 status and TMB status (p=1.0).

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the claimed invention.

1. A method of predicting success of a Urothelial carcinoma (UC) treatment in a patient in need thereof, comprising:
(a) determining the expression of programmed death-ligand 1 (PD-L1) on the patient’s tumor cells (TCs) and immune cells (ICs), and
(b) determining the patient’s tumor mutational burden (TMB),
wherein high PD-L1 expression predicts success of the treatment, and wherein a high TMB further predicts success of the treatment.

2. The method of claim 1, wherein the method further comprises determining if the patient has a somatic mutation in fibroblast growth factor receptor 3 (FGFR3) gene, wherein a lack of a somatic mutation in FGFR3 gene further predicts success of the treatment.

3. The method of claim 1, wherein the high PD-L1 expression comprises expression of PD-L1 on 25% or more of TCs and/or ICs.

4. The method of claim 1, wherein the UC treatment comprises treatment with durvalumab.

5. The method of claim 1, wherein success of treatment is determined by an increase in overall survival as compared to standard of care.

6. The method of claim 1, wherein the patient previously received at least one line of platinum-based chemotherapy.

7. The method of claim 2, wherein determining if the patient has a somatic mutation in FGFR3 gene is determined using the patient’s circulating tumor DNA (ctDNA).

8. The method of claim 1, wherein the UC treatment is a treatment for urothelial carcinoma.

9. A method of predicting success of a urothelial carcinoma (UC) treatment in a patient in need thereof, comprising determining if the patient has a somatic mutation in fibroblast growth factor receptor 3 (FGFR3) gene, wherein a lack of a somatic mutation in FGFR3 gene predicts success of the treatment.

10. The method of claim 9, wherein the UC treatment comprises treatment with durvalumab.

11. The method of claim 9, wherein success of treatment is determined by an increase in overall survival as compared to standard of care.

12. The method of claim 9, wherein the patient previously received at least one line of platinum-based chemotherapy.

13. The method of claim 9, wherein determining if the patient has a somatic mutation in FGFR3 gene is determined using the patient’s circulating tumor DNA (ctDNA).

14. A method of predicting success of a urothelial carcinoma (UC) treatment in a patient in need thereof, comprising determining if the patient has a somatic mutation in at least one of AT-rich interactive domain-containing protein 1A gene (ARID1A) or notch receptor 1 (NOTCH1) gene, wherein a somatic mutation in at least one of ARID 1A or NOTCH1 gene predicts success of the treatment.

15. The method of claim 14, wherein the cancer treatment comprises treatment with durvalumab.

16. The method of claim 14, wherein success of treatment is determined by an increase in overall survival as compared to standard of care.

17. The method of claim 14, wherein the patient previously received at least one line of platinum-based chemotherapy.

18. The method of claim 14, wherein determining if the patient has a somatic mutation in at least one of ARID1A or NOTCH1 gene is determined using the patient’s circulating tumor DNA (ctDNA).

19. A method of treating urothelial carcinoma (UC) in a patient in need thereof, comprising:
(a) determining if the patient has a somatic mutation in fibroblast growth factor receptor 3 (FGFR3) gene; and
(b) treating or continuing treatment if patient lacks a somatic mutation in FGFR3 gene or discontinuing treatment if the patient has a somatic mutation in FGFR3 gene.

20. The method of claim 19, wherein the treatment comprises treatment with durvalumab.

21. A method of treating urothelial carcinoma (UC) in a patient in need thereof, comprising:
(a) determining whether the patient has a somatic mutation in at least one of A-T-rich interactive domain-containing protein 1A gene (ARID1A) or notch receptor 1(NOTCH1) gene; and
(b) treating or continuing treatment if patient has a somatic mutation in at least one of ARID1A or NOTCH1 gene.

22. The method of claim 21, wherein the treatment comprises treatment with durvalumab.
